(19) United States
(12) Patent Application Publication
(10) Pub. No.: US 2026/0260488 A1
(43) Pub. Date: Sep. 3, 2026

(54) Computer Vision Based Two-Stage Surgical Phase Recognition Module

(71) Applicant: VERILY LIFE SCIENCES LLC, Dallas, TX (US)

(72) Inventors: Tomer Golany, Tel-Aviv (IL); Ehud Rivlin, Haifa (IL); Daniel Freedman, Zikhron Yaaqov (IL); Nadav Rabani, New York City, NY (US); Yun Liu, Mountain View, CA (US); Amit Aides, Haifa (IL); Gregory Sean Corrado, San Francisco, CA (US); Boulous Jaber, Haifa (IL); Yossi Matias, Tel-Aviv (IL)

(21) Appl. No.: 18/869,608

(22) PCT Filed: May 24, 2023
(86) PCT No.: PCT/US2023/023326

§ 371 Date: Nov. 26, 2024

Related U.S. Application Data
(60) Provisional application No. 63/345,990, filed on May 26, 2022.

(51) Int. Cl. G06V 20/40; G06V 10/62; G06V 10/774; G06V 10/82

(52) U.S. Cl. CPC G06V 20/41; G06V 10/62; G06V 10/774; G06V 10/82; G06V 20/44; G06V 20/46; G06V 2201/03

(57) ABSTRACT

A system and method of identifying an adverse event using a video of a surgery and a two-stage surgical phase recognition module. The method includes receiving, by the module, a video of the surgery, where the video comprises a sequence of video frames. The module comprises a first stage that includes a neural network and a second stage that includes a multi-stage temporal convolution network. The method includes extracting, using the first stage, visual information content of a single frame based on the single frame; identifying, using the second stage, surgical phases captured in the frames of the video based on the visual information content from the first stage; and identifying, using the identified surgical phases, an adverse event during the surgery. An adverse event includes the omission of a surgical phase and an injury to the patient. The identification can occur in real-time or near-real-time.

### PRIORITY STATEMENT & CROSS-REFERENCE TO RELATED APPLICATIONS

This application is a U.S. national phase application of International Application No. PCT/US 2023/023326 filed May 24, 2023, which designates the U.S. and claims the benefit of U.S. Application No. 63/345,990 filed May 26, 2022, both of which are hereby incorporated by reference in their entirety.

### TECHNICAL FIELD

The present disclosure relates generally to the use of a computer vision based two-stage surgical phase recognition module to identify surgical phases in surgeries.

### BACKGROUND

Currently, different types of surgeries are saved as a video file. After the surgery, expert surgeons manually review the video file to perform a variety of tasks, such as auditing quality measures, analyzing and recording any adverse events, referencing the video file for educational purposes, evaluating the surgical performance, etc. This manual review is time consuming and does not prevent an adverse event from happening during the surgery. Instead, the review only detects and records an adverse event that has already occurred. Using an artificial intelligence (“AI”) model that is, or includes, a two-stage surgical phase recognition (“SPR”) module, however, allows for the video of the surgery to be analyzed in real-time or near-real-time to identify different phases of the surgery as well as prevent, or at least reduce the likelihood of, an adverse event. Moreover, a two-stage SPR module that monitors surgery is also capable of predicting the completion time of the surgery, therefore improving scheduling of the operating room(s) and staff.

### SUMMARY

The present disclosure describes a system configured to automatically identify a surgical phase of a surgery captured in a video of the surgery, with the surgery including a plurality of sequential surgical phases. The system includes a non-transitory computer readable medium having stored thereon a plurality of instructions, and when the instructions are executed with one or more processors, the following steps are executed: receiving, by a two-stage surgical phase recognition (“SPR”) module, the video of the surgery; wherein the two-stage SPR module comprises a first stage and a second stage; extracting, using a neural network that forms the first stage, visual information content of a single frame based on the single frame; and identifying, using a multi-stage temporal convolution network that forms the second stage, surgical phases captured in the frames of the video based on the visual information content from the first stage; and wherein the two-stage SPR module has been trained using videos of surgeries involving complex anatomies and videos of surgeries involving adverse events.

The present disclosure also describes a system configured to identify an adverse event during a surgery of a patient based on a video of the surgery. The surgery is on a patient and comprises a plurality of sequential surgical phases. The video includes a sequence of video frames. The instructions are executed with one or more processors so that the following steps are executed: receiving, by a two-stage surgical phase recognition (“SPR”) module, the video of the surgery; wherein the two-stage SPR module comprises a first stage and a second stage; extracting, using a neural network that forms the first stage, visual information content of a single frame based on the single frame; identifying, using a multi-stage temporal convolution network that forms the second stage, surgical phases captured in the frames of the video based on the visual information content from the first stage; and identifying, using the two-stage SPR module and the identified surgical phases, an adverse event during the surgery; wherein the adverse event comprises at least one of: the absence of a surgical phase in the plurality of sequential surgical phases; and an injury to the patient.

The present disclosure also describes a method of identifying an adverse event using a video of a surgery and a two-stage surgical phase recognition (“SPR”) module. The method includes receiving, by the two-stage SPR module, a video of the surgery; wherein the video comprises a sequence of video frames; wherein the surgery is on a patient and comprises a plurality of sequential surgical phases; and wherein the two-stage SPR module comprises a first stage and a second stage; extracting, using a neural network that forms the first stage, visual information content of a single frame based on the single frame; identifying, using a multi-stage temporal convolution network that forms the second stage, surgical phases captured in the frames of the video based on the visual information content from the first stage; and identifying, using the two-stage SPR module and the identified surgical phases, an adverse event during the surgery; and wherein the adverse event comprises at least one of: the absence of a surgical phase in the plurality of sequential surgical phases; and an injury to the patient.

### BRIEF DESCRIPTION OF THE DRAWINGS

FIG. 1 is a diagrammatic illustration of a surgical system operably coupled to a two-stage surgical phase recognition (“SPR”) module, according to an example embodiment.

FIG. 2 is a simplified diagram illustrating the structure of the two-stage SPR module of FIG. 1, according to an example embodiment.

FIG. 3 is a diagrammatic illustration of the two-stage SPR module of FIG. 1, according to an example embodiment.

FIG. 4 is a flow chart illustration of a method of training the two-stage SPR module of FIG. 1, according to an example embodiment.

FIG. 5 is a table detailing the general characteristics of a collected data set used to train and test the two-stage SPR module of FIG. 1, according to an example embodiment.

FIG. 6 is a table detailing the complexity level of each procedure within the collected data set, according to an example embodiment.

FIG. 7 is a table that includes example mapping between each procedure to its complexity level, according to an example embodiment.

FIG. 8 is a chart illustrating the evaluation of the per-phase confusion matrix, according to example embodiment.

FIG. 9 is a graph illustrating the mean accuracy of a portion of the two-stage SPR module, according to an example embodiment.

FIG. 10 is a graph illustrating overall accuracy of a portion of the two-stage SPR module on the test set, relative to adverse events in laparoscopic cholecystectomy (LC) procedures, according to example embodiment.

FIG. 11 is a graph illustrating the overall accuracy of a portion of the two-stage SPR module, according to both the source hospital as well as the average complexity level, according to example embodiment.

FIG. 12 is a graph illustrating the average accuracy of a portion of the two-stage SPR module during testing, according to an example embodiment.

FIG. 13 is a flow chart illustration of a method of using the two-stage SPR module of FIG. 1, according to an example embodiment.

### DETAILED DESCRIPTION

An AI system disclosed herein and that recognizes surgical phases may be used for many important tasks like quality measures, adverse events recording and analysis, education, statistics, surgical performance evaluation and more. Currently, these tasks are performed manually in a time-consuming fashion by expert surgeons. Use of the described system during surgery would further enable real-time monitoring and assisted decision making, which may increase safety and improve patient outcomes. For example, a real-time assistive system can alert the surgeon to an incorrect plane of dissection, a wrong maneuver, or an upcoming complication. The system may also be used as a context-aware decision support system by providing early warnings in case of misorientation or other unexpected events. As a specific example in laparoscopic cholecystectomy (LC), achieving the Critical View of Safety (CVS) is the recommended strategy for minimizing the risk of Bile Duct Injury (BDI), therefore the described system, which is capable of detecting and verifying that CVS has been achieved, may reduce the risk of injuries. The described system can also optimize operating room (OR) utilization and staff scheduling and provide administrative assistance by analyzing the progress of an operation and more accurately predicting the time required for procedure completion.

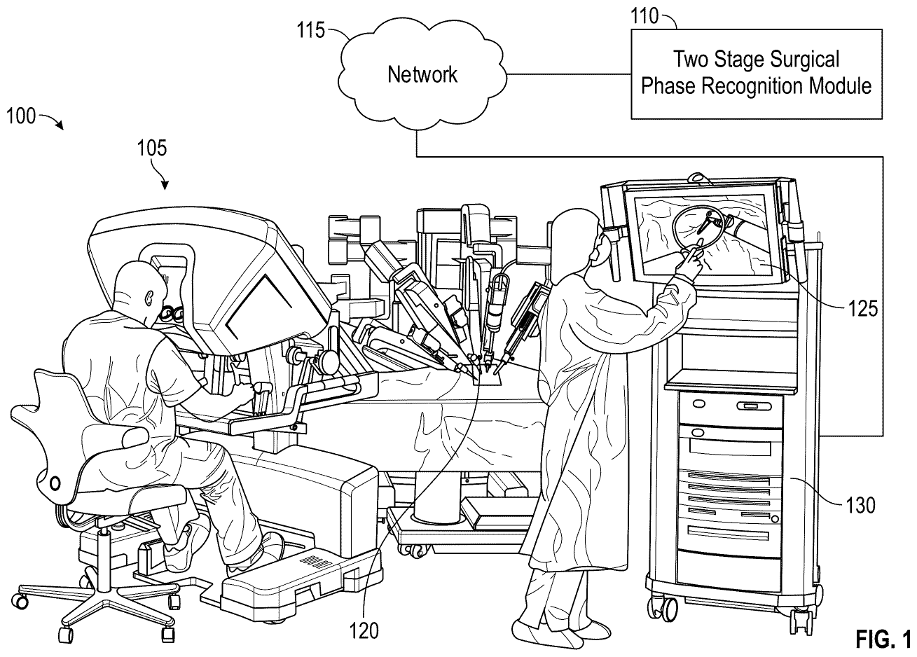

In an example embodiment, as illustrated in FIG. 1, a system is generally referred to by the reference numeral 100 and includes a surgical system 105 that is in communication with an AI model that is a two-stage SPR module 110. As used herein, the term “module” and “model” are interchangeable and may include hardware or software-based framework that performs one or more functions. In some embodiments, the two-stage SPR module 110 is in communication with the surgical system 105 via a network 115. Generally, the system 100 allows for a surgery to be recorded and the video to be analyzed, by the two-stage SPR module 110, at a later date or analyzed in real-time or near-real-time so that feedback regarding the surgery can be provided during the surgery. In some embodiments, near-real-time refers to the time delay introduced, by automated data processing or network transmission, between the occurrence of an event and the use of the processed data, such as for display or feedback and control purposes. In some embodiments, near-real-time includes an intentional time delay to allow for the receipt/creation of subsequent, additional video frames (compared to a target video frame) to be considered by the two-stage SPR module 110 when analyzing the target video frame.

Regarding the surgical system 105, the surgical system 105 generally includes at least one camera 120 for recording a surgery, a user interface (“UI”) 125, and a computing device 130. In some embodiments, the two-stage SPR module 110 is stored and executed by the computing system 130. In other embodiments and as illustrated, the two-stage SPR module 110 may be physically separated from the computing system 130 and hosted at another location but accessible to the computing system 105 via the network 115.

Regarding the network 115, the network 115 includes the Internet, one or more local area networks, one or more wide area networks, one or more cellular networks, one or more wireless networks, one or more voice networks, one or more data networks, one or more communication systems, and/or any combination thereof.

### The Two-Stage SPR Module

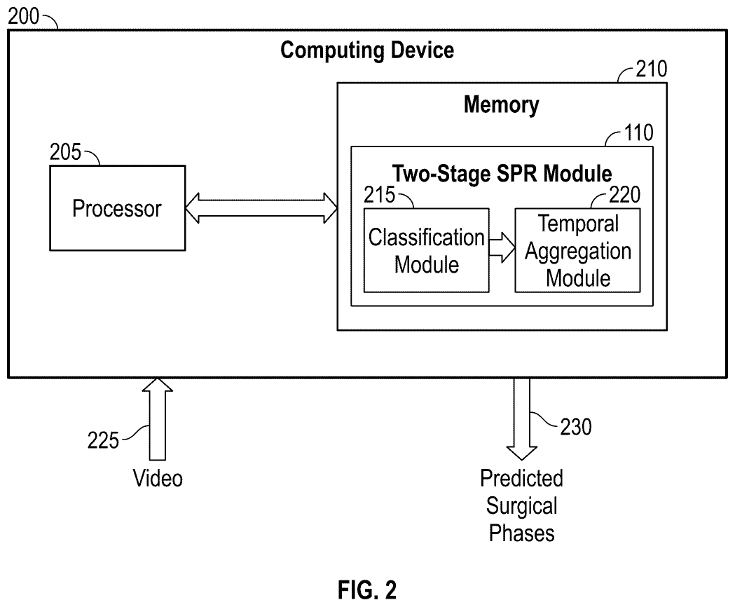

FIG. 2 is a simplified diagram of a computing device 200 implementing the two-stage surgical phase recognition (“SPR”) process, according to some embodiments. As shown in FIG. 2, the computing device 200 includes a processor 205 coupled to a memory 210. Operation of the computing device 200 is controlled by the processor 205. And although the computing device 200 is shown with only one processor 205, it is understood that the processor 205 may be representative of one or more central processing units, multi-core processors, microprocessors, microcontrollers, digital signal processors, field programmable gate arrays (FPGAs), application specific integrated circuits (ASICs), graphics processing units (GPUs), tensor processing units (TPUs), and/or the like in the computing device 200. The computing device 200 may be implemented as a stand-alone subsystem, as a board added to a computing device, and/or as a virtual machine.

The memory 210 may be used to store software executed by the computing device 200 and/or one or more data structures used during operation of the computing device 200. The memory 210 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

The processor 205 and/or the memory 210 may be arranged in any suitable physical arrangement. In some embodiments, the processor 205 and/or the memory 210 may be implemented on the same board, in the same package (e.g., system-in-package), on the same chip (e.g., system-on-chip), and/or the like. In some embodiments, the processor 205 and/or the memory 210 may include distributed, virtualized, and/or containerized computing resources. Consistent with such embodiments, the processor 205 and/or the memory 210 may be located in one or more data centers and/or cloud computing facilities.

In some examples, the memory 210 may include non-transitory, tangible, machine readable media that includes executable code that when run by one or more processors (e.g., the processor 205) may cause the one or more processors to perform the methods described in further detail herein. For example, as shown, the memory 210 includes the two-stage SPR module 110 that may be used to implement and/or emulate the systems and models, and/or to implement any of the methods described further herein.

The two-stage SPR module 110 includes two sub-modules, the classification module 215 and the temporal aggregation module 220. The classification module 215 and the temporal aggregation module 220 are operated sequentially to receive input (e.g., a streaming video 225), compute and exchange intermediate parameters or variables, and generate a final output of identification of predicted surgical phases 230. In some examples, the two-stage SPR module 110 and the two sub-modules 215 and 220 may be implemented using hardware, software, and/or a combination of hardware and software.

As shown, the computing device 200 receives as input the streaming video 225, which is provided to the two-stage SPR module 110. For example, the input streaming video 225 may include a real-time video feed from the surgical system 105. The two-stage SPR module 110 operates on the input video stream 225 to detect, via the classification module 215, a category of an action in the video stream to predict a surgical phase for each frame, and finalizing, via the temporal aggregation module 220, predictions of the surgical phases for each frame.

In some embodiments, the classification module 215 is built on a deep residual convolutional neural network (“RCNN”), and the temporal aggregation module 220 includes a Multi-Stage Temporal Convolution Network (MS-TCN).

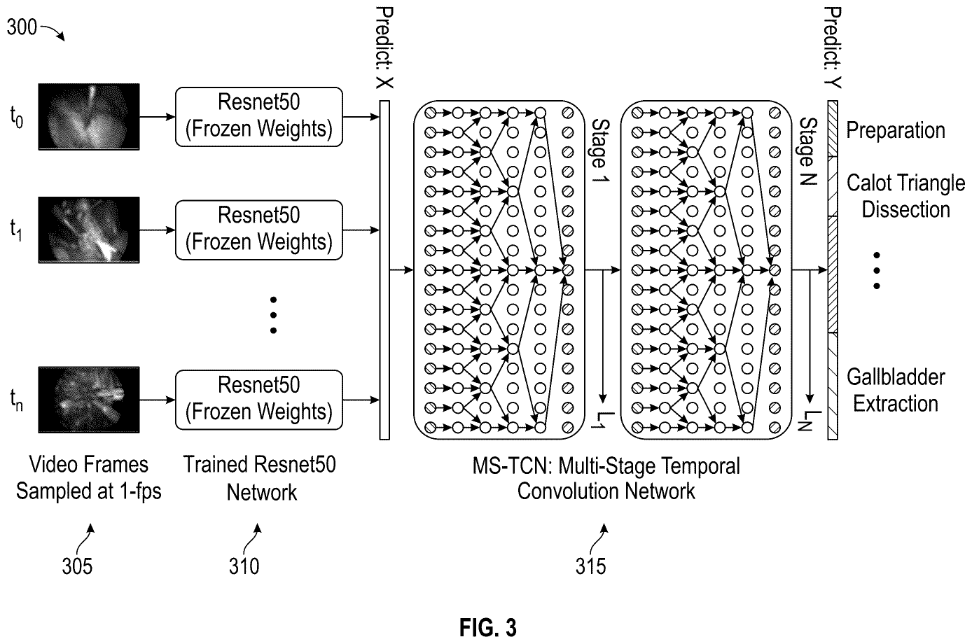

FIG. 3 illustrates a diagrammatic illustration of a data flow, referenced by the numeral 300, associated with the two-stage SPR module 110. At step 305, the LC video is processed at 1 frame per second (fps) to create frames t₀, t₁ . . . tₙ. However, the LC video may be processed at greater than or less than 1 frame per second. At step 310, each frame is fed into a deep convolutional neural network, which is ResNet50 in this example. The ResNet50 model is trained to classify each frame's associated surgical phase independently. In some embodiments, the first stage is temporal-agnostic such that the first stage extracts visual features from single frames, without any temporal context. As such, the visual information extracted by the neural network is temporal-agnostic.

As indicated in FIG. 3, following training, the last prediction layer of the ResNet50 is removed, and all the network parameters are frozen (not trainable subsequently). The initial prediction from the first stage is used as input to the second stage. For each frame, the ResNet50 produces a feature vector, which expresses the visual information content of the frame as a lower dimensional (compared to the original frame) numerical “feature vector.” At step 315, all feature vectors are combined to form a sequence of feature vectors representing the entire LC video. This sequence is an input to the MS-TCN model, which consists of temporal convolution layers with a dilation rate that increases across layers. In one embodiment, the temporal aggregation module 220 includes a MS-TCN architecture with five stages, with each stage containing 19 dilated convolution layers, where the dilation factor is doubled at each layer and dropout is used after each layer. In some embodiments, all layers have 64 filters, each of size 3 and a ReLU (rectified linear) activation. In some embodiments, residual connections are used to facilitate gradient flow. To get the probabilities for the output phase for each frame, a 1×1 convolution is applied over the output of the last dilated convolution layer followed by a softmax activation.

In some embodiments, an Adam optimizer, or Adam optimization algorithm, is used with a learning rate of 0.0001 to minimize or reduce the average cross-entropy loss. The temporal aggregation module 220 allows the module to capture long-range time dependencies and recognize temporal phase segments. The temporal convolution layers capture temporal connections, and the increasing dilation setup enables the capturing of long-term temporal dependencies. The final layer of the MS-TCN model outputs the surgical phase prediction for each frame in the video. As illustrated in FIG. 3, the phrases include “preparation”, “calot triangle dissection”, and “gallbladder extraction” but others are considered.

### Training the Two-Stage SPR Module

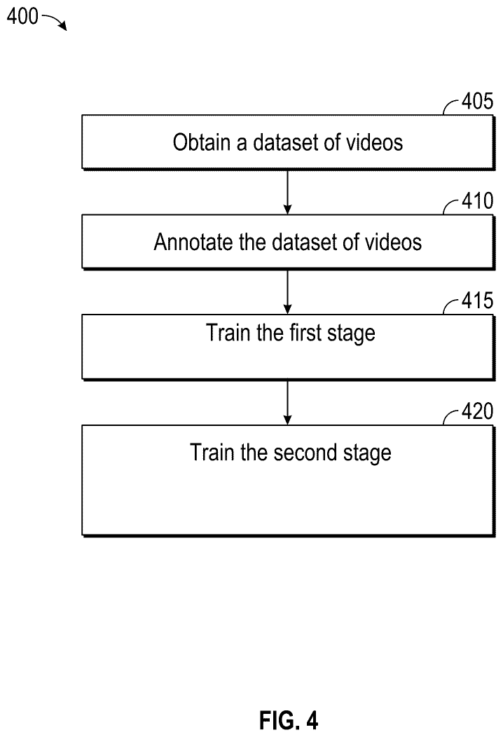

The two-stage SPR module 110 is trained to recognize and identify phases of both straightforward and complicated procedures. While the example described herein discusses a laparoscopic cholecystectomy (LC), the two-stage SPR module 110 can be configured for and trained for other types of surgeries, whether laparoscopic or open. In an example embodiment, as illustrated in FIG. 4 with continuing reference to FIGS. 1-3, a method 400 of training the system includes obtaining a dataset of videos at step 405, annotating the dataset of videos at step 410, training the first stage at step 415, and training the second stage at step 420.

In an example embodiment and at step 405, a dataset of videos or video files is obtained. The dataset of videos may be collected from a number of hospitals and surgeons with specific criteria regarding the type of surgery performed in the video, age of the patient, etc. In one example, 448 cholecystectomy videos of laparoscopic cholecystectomy for biliary colic or acute and chronic cholecystitis in patients 18 years of age or older was obtained. The dataset included 368 videos collected from four hospitals in Israel and 80 videos from the publicly available Cholec80 dataset collected from a hospital in France. The videos were recorded between Nov. 1, 2010, and Oct. 1, 2020.

After the videos are obtained, the videos are annotated at the step 410. Any videos that cannot be annotated by surgeons are excluded from the dataset. In some embodiments and in the example, the phases and annotation process were determined via consensus of a group of experienced senior surgeons. Each video is annotated according to identified phases of the surgery, such as for example the following phases for laparoscopic cholecystectomy: 1) trocar insertion, 2) preparation, 3) Calot triangle dissection, 4) clipping and cutting, 5) gallbladder dissection, 6) gallbladder packaging, 7) cleaning and hemostasis, and 8) gallbladder extraction. Additionally, two special phases may be used in annotation. First, segments in which the camera was not placed inside the body were annotated as “out of body.” Second, segments in which the camera was not focused on tools and no surgical action was being performed were annotated as “idle.” To analyze the ability of the two-stage SPR module 110 to recognize the major surgical phases in videos of abnormal or challenging LC procedures, a set of important adverse events were also identified by the expert surgeons. The adverse events that were annotated included: 1) major bleeding, 2) gallbladder perforation, 3) major bile leakage, and 4) incidental finding.

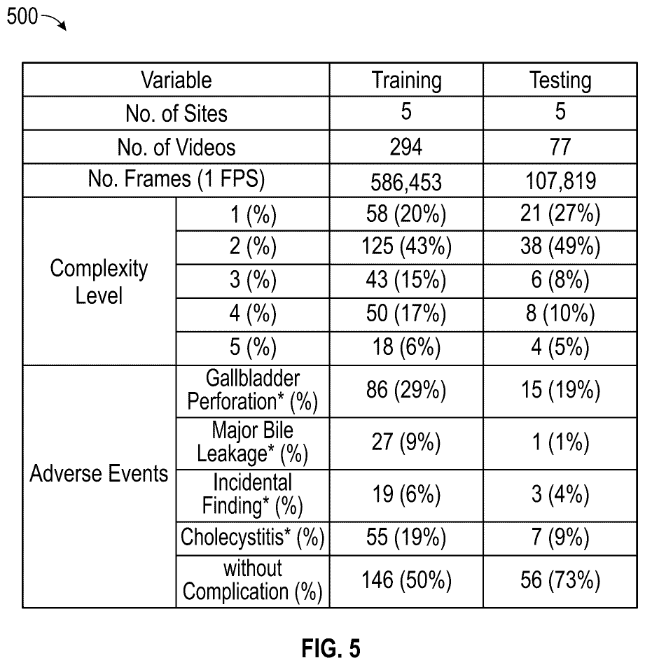

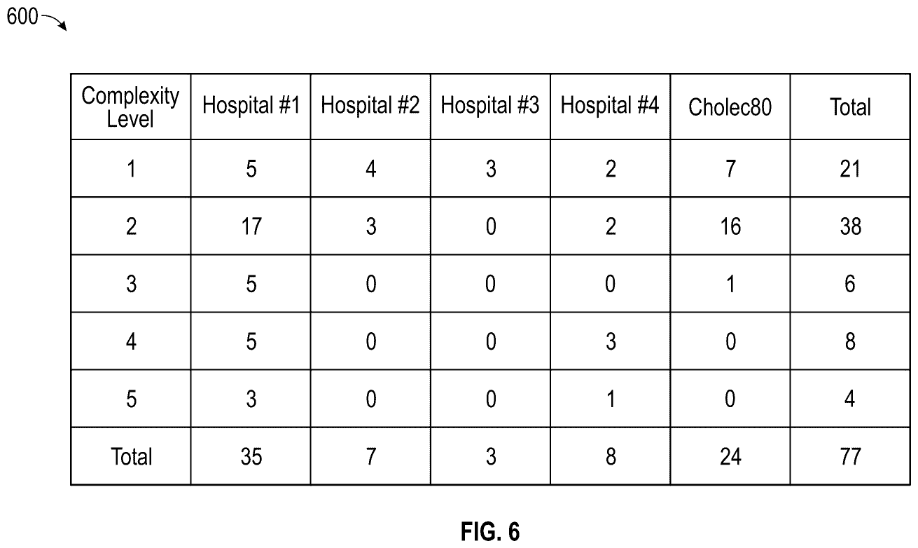

In addition to annotating the phases and adverse events described above, annotations may also be collected for the complexity level of each procedure. The complexity level may be scored on a scale of 1-5 based on intraoperative parameters. The factors to determine the complexity level included: state of the gallbladder (based on the Parkland Grading Scale for grading still images of Cholecystitis), presence of intra-abdominal adhesions, normality of anatomy, duct closure device utilized, performance of intraoperative cholangiography, partial or open cholecystectomy requirements and intraoperative adverse events. After excluding videos that could not be annotated consistently by surgeons, 371 videos remained and were used for the example. FIG. 5 includes a table 500 that categorizes the dataset. FIG. 6 includes a table 600 with a breakdown of complexity level of each procedure within the test-set, per hospital institution. The dataset was split in an 80:20 ratio respectively for training and testing the two-stage SPR module 110, with the splits stratified by surgical complexity, institution, and adverse events during surgery. The splitting was performed on a per-case rather than a per-frame level. That is, frames from a video in the training set did not appear in the test set

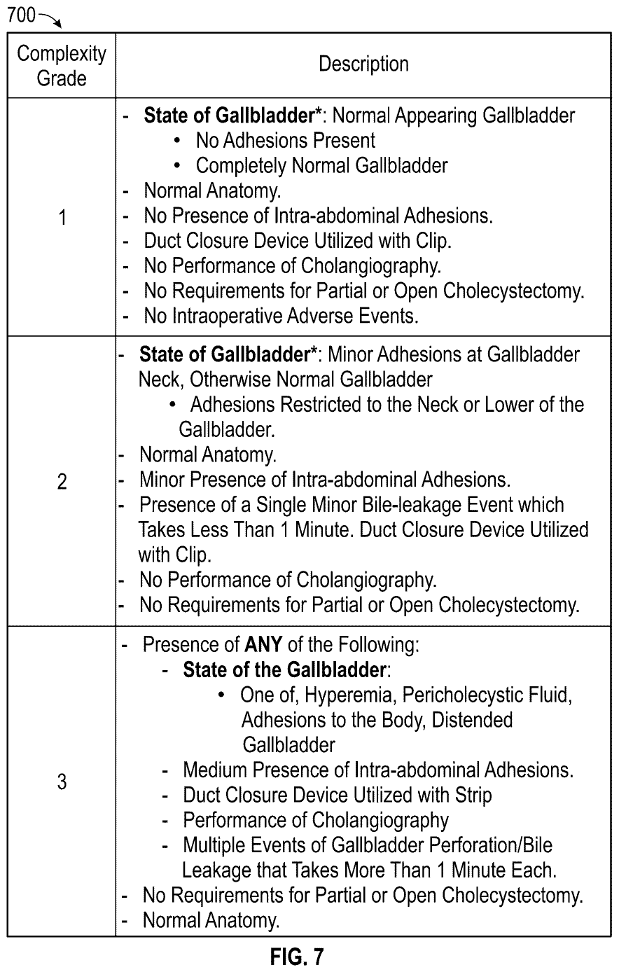

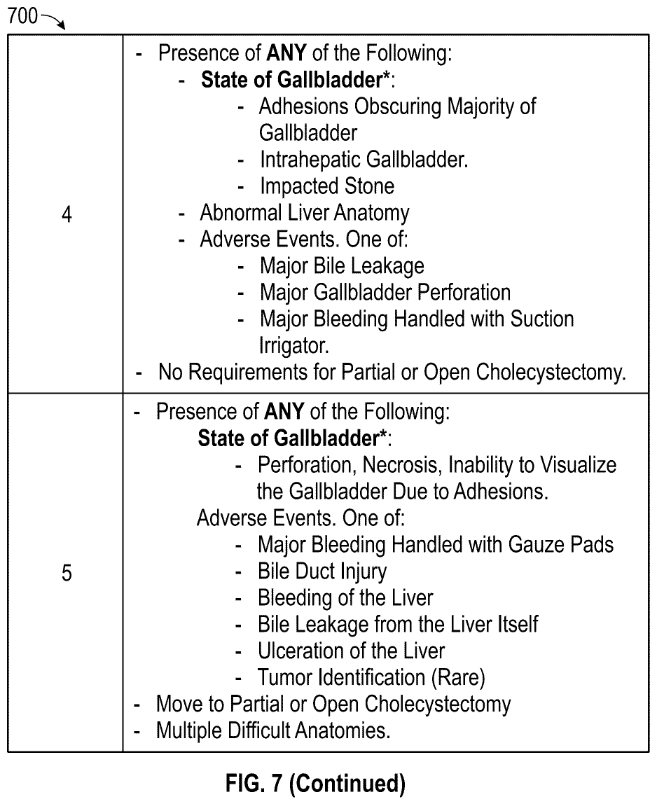

FIG. 7 provides a table 700 that details an example mapping between each procedure to its complexity level. The annotations of complexity levels and complications were used for assessing the two-stage SPR module's ability to accurately recognize the surgical phases in complex LC procedures. The videos were also annotated to note if the CVS was achieved during the Calot triangle dissection phase. The CVS phase is defined when the neck (infundibulum) of the gallbladder is dissected off the liver bed, to achieve conclusive identification of the two structures to be divided: the cystic duct and cystic artery. In the classic view the liver is seen through the Calot triangle. The annotations were performed by 13 surgeons with at least 4 years of experience (median: 7, range: 4-15) in general surgery. Annotator training included understanding the definition of each phase and adverse event; learning how to indicate the start and end of each phase; and becoming familiar with the annotation software. To validate the quality of the annotations, each video was annotated by two annotators, and the inter-rater agreement score between them was calculated. The inter-rater agreement score was defined as the number of frames annotated with the same phase label by the two annotators, divided by the total number of annotated frames in the video. Videos with an agreement score below 80% (n=77) were excluded to arrive at the final set of 371 videos in the dataset.

At step 415, the first stage is trained using a portion of the dataset. The first stage, which includes the RCNN, extracts features from the video frames. While classical classification models focus on extracting hand-crafted features (colors, corners, edges, etc.), and combining them as inputs to supervised machine learning models, deep neural networks learn the features by themselves from the raw data. The extracted features are thus optimized to improve classification performance. The deep residual convolutional neural network, ResNet50, was applied to extract features from LC frames. Given a single frame taken from a cholecystectomy procedure as input, the ResNet50 model outputs a vector with a probability score for each phase. The first stage predicts the phase of the video frame based on the extracted features of a single frame. When the training of the ResNet50 model is completed, the network weights are frozen, and the last prediction layer is removed. The resulting frozen network is used to extract feature vectors from the raw cholecystectomy frames.

At step 420, the second stage is trained. For the temporal aggregation stage, a temporal convolution network is utilized. In one embodiment and as noted above, the MS-TCN network is utilized and consists of multiple stages, where each stage is composed of dilated temporal convolution blocks. The dilated temporal convolution blocks enable the module to gain a large temporal receptive field with fewer parameters, which eases learning of temporal dependencies over the entire cholecystectomy video. Each stage of the network outputs an initial prediction that is refined by the next one. The MS-TCN network receives as input a sequence of feature-vectors (each of which is a single frame processed by the ResNet50 model in the first stage) which represent a complete cholecystectomy video; and outputs a phase prediction for each feature-vector in the input sequence.

In one embodiment, the MS-TCN model used is non-causal, that is, the prediction of the phase at timestep t, depends on both past and future frames to optimize for overall accuracy, because information about subsequent steps in “future frames” should help categorize the current frame. In some embodiments, only a few future frames are used to result in minimal increases in latency. In some embodiments, the training runs for 50 epochs and the final model is the one with the best validation results during the optimization process.

Batch size is a tuned hyperparameter, and all training videos were resampled to 1 frame/second, and zero-padded to the longest video in each batch. The hyperparameters were chosen among the following options: number of dilated layers in each stage (5, 7, 10, 12, 15, 17, 19, 20), number of stages (1, 2, 3, 4, 5), batch size (4, 8, 16, 32), learning rate (0.01, 0.001, 0.0001), optimization algorithms of Stochastic gradient descent (“SGD”), rate-monotonic scheduling (“RMS”), Adaptive Moment Estimation (“ADAM”), dropout rate (0, 0.5, 0.8). In addition, and in one example, an additional smoothing loss was added and different feature extractor models (Inception V3, 13D) were used.

### Evaluating the Two-Stage SPR Module

In one example, the module was evaluated on the test set using the accuracy metric. Accuracy quantifies the fraction of frames with correctly classified phases and is defined as the number correctly classified frames divided by the total number of evaluated frames. On average, a small fraction (0.16%) of the frames in each video was not annotated due to difficulties in selecting precise start/end frames for annotation in a way that eliminates unannotated gaps. The accuracy was calculated on both the first-stage (ResNet50) model alone and the second-stage (MS-TCN) model. This frame-level accuracy per video was then averaged over all videos to ensure each video was equally weighted. For error bars, the 95% empirical confidence interval (CI) was computed by bootstrapping across videos. To place the model's accuracy in perspective, each video was annotated by a second surgeon. The inter-surgeon agreement was then computed by evaluating the second surgeon's accuracy per-video against the first, and similarly averaging across all videos. The first stage (ResNet50) model achieved overall classification accuracy of 78% on the test set. The second stage (MS-TCN) model, which incorporates temporal information across the whole video, obtained higher accuracy, reaching 89% accuracy on the test set.

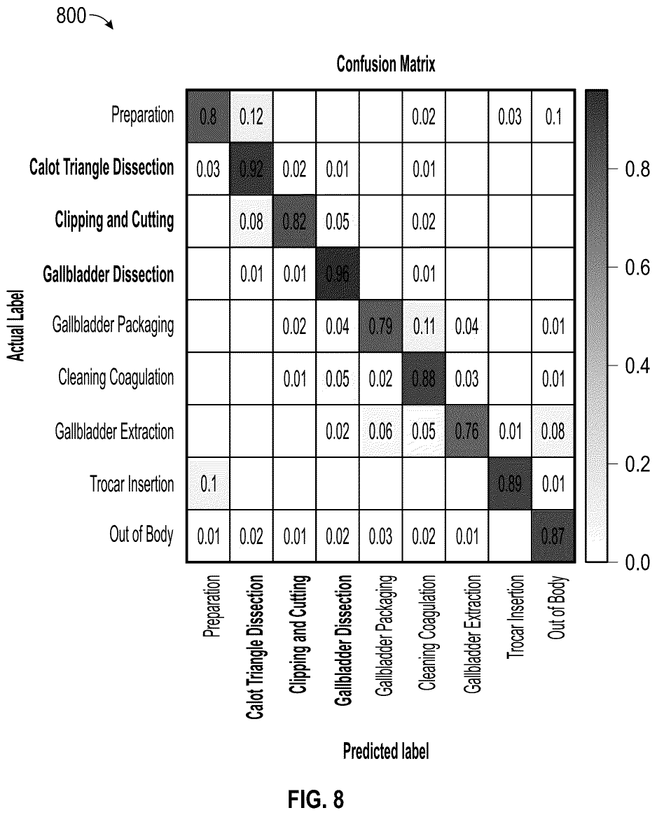

FIG. 8 shows a chart 800 illustrating the evaluation of the per-phase confusion matrix, reached by the full two-stage module 110, with the critical phases in an LC procedure being the Calot triangle dissection phase, the clipping and cutting phase, and the gallbladder dissection phase. The per-phase calculation was performed across all frames per video, and then averaged over all videos in the test set. As illustrated, the model successfully detected the most critical phases—Calot triangle dissection, clipping and cutting, and gallbladder dissection phase—with accuracies of 92%, 82%, and 96%, respectively. For the preparation phase, the model reached 80% accuracy; however, 12% of these preparation frames were incorrectly predicted as part of the Calot triangle dissection phase instead. These erroneous predictions are distributed along the transition between the two phases. As previously noted, the complexity level of each LC video in the dataset was annotated on a scale of 1-5.

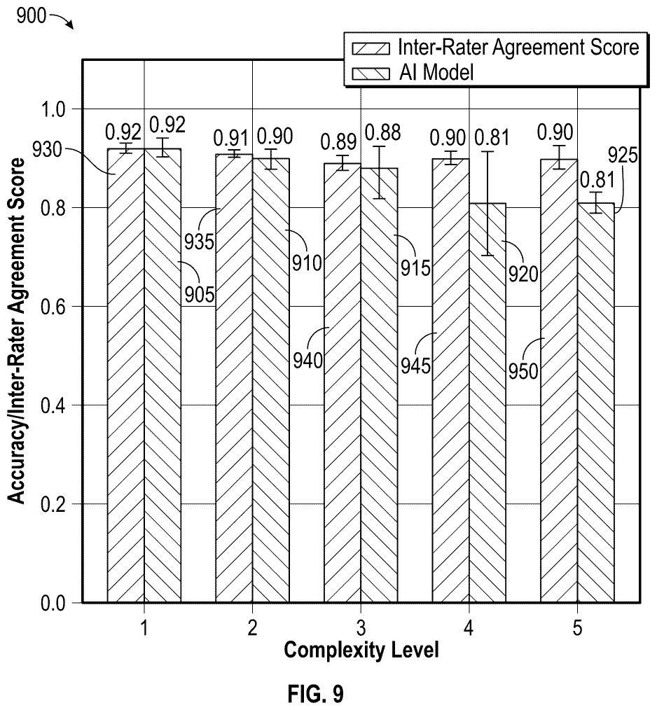

FIG. 9 illustrates a graph 900 showing the mean accuracy of our MS-TCN model with bars 905, 910, 915, 920, and 925, and the inter-rater score agreement with bars 930, 935, 940, 945, and 950 on the test set videos relative to their complexity. As the complexity increases from 1 to 3, the model's accuracy linearly decreases from 92% to 88%. At complexity levels 4 and 5, the model accuracy was 81%. The inter-rater agreement (between expert surgeons) ranged from 92% on LC procedures with a complexity level of 1 to 90% on LC procedures with a complexity level of 5. For simple LC procedures, the two-stage SPR module 110 has an ability equal to a surgeon in the recognition of surgical phases. In some embodiments, adverse events during LC procedures affect the ability of the two-stage SPR module 110 to recognize the surgical phases.

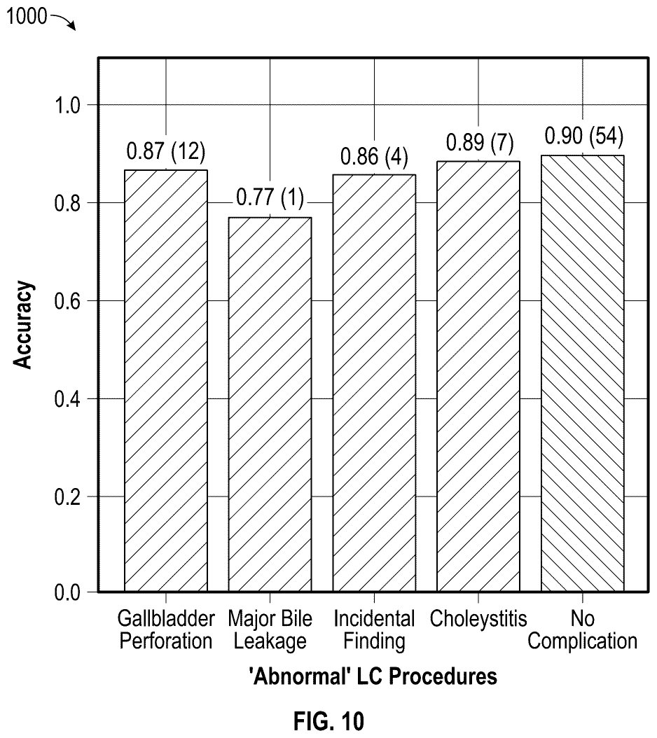

FIG. 10 is a graph 1000 that shows overall accuracy of the module 110 on the test set, relative to adverse events in LC procedures. The module 110 reached an accuracy of 87% in videos with a gallbladder perforation event, 77% on a single video with a major bile leakage event, 86% on videos with an incidental finding, and 89% on procedures with cholecystitis. On videos without adverse events or “No Complication”, the model reached a mean accuracy of 90%. Thus, as expected, in LC procedures with adverse events, the two-stage SPR module 110 attained a lower accuracy. Surgical procedures, such as LC procedures, are different in different hospitals. As previously described, the dataset in the example was composed of procedures from five hospitals. Some variation was noted in the instruments used, as well as in surgical technique, which made the task of identifying the surgical phases more challenging.

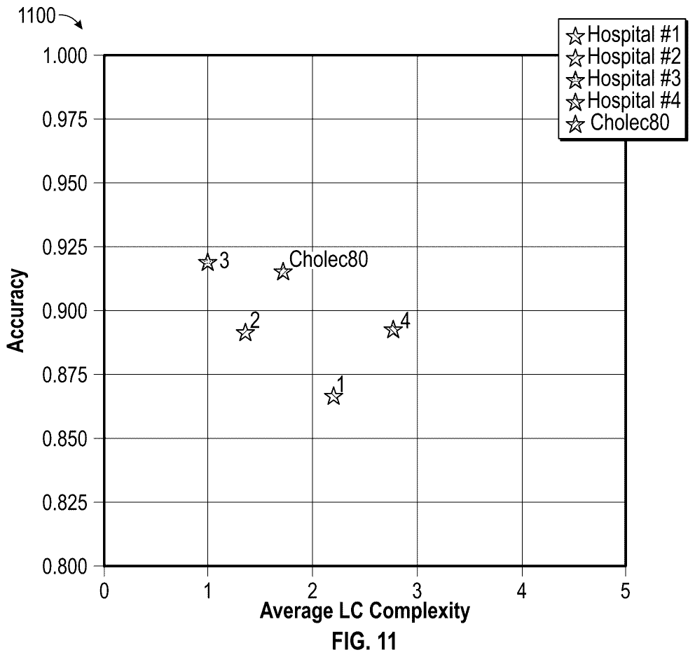

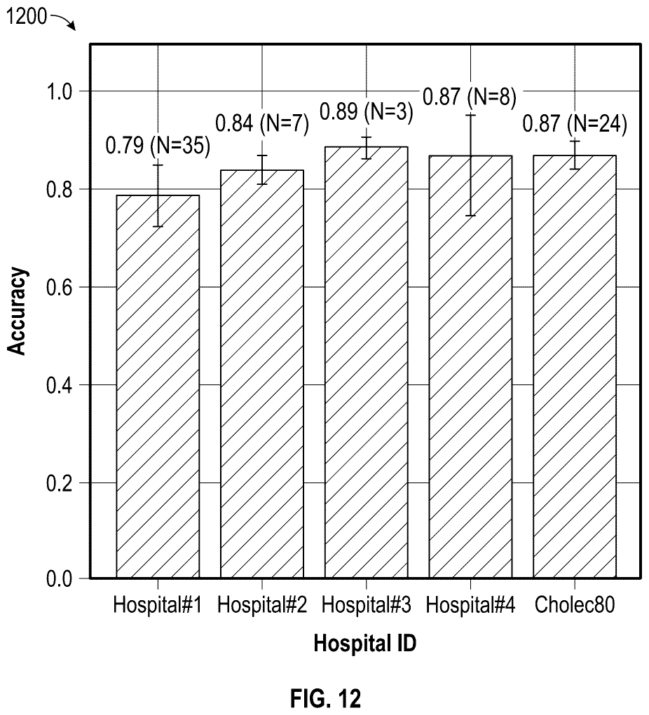

FIG. 11 includes a graph 1100 that shows the overall accuracy of the module 110, according to both the source hospital as well as the average complexity level. Each marker in the graph 1100 represents a different hospital source. The x-value of each marker is the average complexity level of LC procedures for the given source hospital. The y-value of each marker is the average accuracy achieved by the two-stage SPR module 110 on LC procedures for the given source hospital. The model attained an accuracy of 86% in videos from hospital #1, 89% on videos from hospital #2, 91.5% on videos from hospital #3, and 89% on videos from hospital #4. On videos from the Cholec80 dataset our model reached an accuracy of 91.4%. To understand how effectively the two-stage SPR module 110 generalizes to various hospitals, the two-stage SPR module 110 was trained on four of the hospitals and tested it on the fifth. This experiment was repeated five times, where each time a different hospital was set aside as the test set (with the remaining four used as the train set). FIG. 12 includes a graph 1200 that shows the average accuracy of the model for each experiment. The model attained an accuracy of 79% in videos from hospital #1, 84% on videos from hospital #2, 89% on videos from hospital #3, and 87% on videos from hospital #4. Using the four hospitals to train the two-stage SPR module and testing it on the Cholec80 dataset, the two-stage SPR module 110 reached an accuracy of 87%.

As described, the system 100 includes a two-stage SPR module 110 to automate the task of phase recognition in LC. The example model successfully detected surgical phases with an overall accuracy of 89%, comparable to the average agreement between surgeon annotators (i.e., 90%), including successful detection even in procedures with adverse events like major bleeding, major bile leakage, major duct injury and gallbladder perforation.

The detection of surgical phases is more critical for certain phases than it is for others. For example, successful identification of the Calot triangle dissection phase, confirmation of the critical view of safety (CVS), or the clipping and cutting phase, are of utmost importance for the patient's safety, while misrecognition of the gallbladder extraction phase is less important and will have a much lower impact on patient safety. As shown, the model 110 was able to reach a very high accuracy (i.e., 92%) in the Calot Triangle Dissection phase that supports CVS.

In some embodiments, higher complexity levels of LC procedures were associated with both lower accuracy on the part of the two-stage SPR module 110, as well as lower inter-rater agreement between surgeons. On less complex LC videos, the two-stage SPR module 110 achieved an overall accuracy of 92%, equal to the inter-surgeon agreement score. By contrast, in complex LC videos, the annotators reached an average agreement score of 90% compared to 81% by the model 110. Importantly however, the accurate identification of the Calot triangle dissection phase was unaffected in complex videos (92%). Furthermore, the performance of the two-stage SPR module 110 remained high in the presence of adverse events, indicating an overall robustness to adverse events during LC procedures. As mentioned, LC videos from 5 hospitals were used in the example, and as expected, some variation in the surgical technique and type of instruments used was noted. Interestingly, such variations did not interfere with the accuracy of the two-stage SPR module 110 in phase recognition reaching 80-87% overall accuracy reflecting the system's flexibility and reliability.

### Example Uses of the Trained Two-stage SPR Module

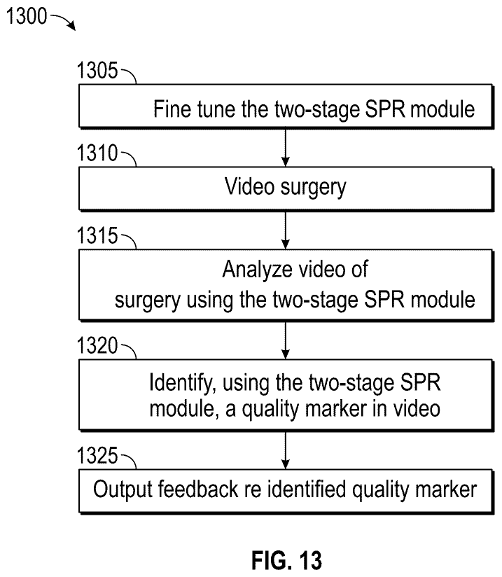

In an example embodiment, as illustrated in FIG. 13 with continuing reference to FIGS. 1-12, a method 1300 of using the system 100 includes fine tuning the trained two-stage SPR module 110 at step 1305, videoing a surgery at step 1310, analyzing the video of the surgery using the trained two-stage SPR module 110 at step 1315, identifying, using the trained two-stage SPR module, a quality marker in the video at step 1320, and outputting feedback based on the identified quality marker at step 1325.

In some embodiments, the trained two-stage SPR module 110 is fine-tuned at the step 1305. In some instances, the two-stage SPR module 110 is stored in a computing system located in one hospital and is expected to analyze videos performed at that one hospital. When the two-stage SPR module 110 is expected to analyze videos associated with one hospital, the two-stage SPR module 110 can be fine-tuned using a training set associated with the one hospital to improve the accuracy of the two-stage SPR module 110. Fine tuning the two-stage SPR module 110 includes obtaining a dataset of videos specific to the hospital and retraining the two-stage SPR module 110 using that dataset. The retraining can include retraining the last prediction layers of the neural network using the dataset of videos specific to the hospital. Generally, the fine tuning improves accuracy because videos of the same surgery can include differences relating to the cameras used, the tools used, etc. As such, retraining the two-stage SPR module 110 with videos specific to the hospital in which the two-stage SPR module 110 will be used improves the accuracy of the two-stage SPR module 110. Retraining may include similar steps to the initial training but for the use of a dataset of videos specific to the hospital.

In some embodiments, a surgery is created at the step 1310. The camera 120 is used to video, stream, capture, or otherwise create the video of the surgery during the step 1310.

In some embodiments, the video of the surgery is analyzed using the model 110 at the step 1315. Similar to the training of the module 110, the video is received and features are extracted by the first stage. The first stage makes a surgical phase prediction based on the features extracted by the first stage. The output of the neural network, or first stage, comprises a sequence of feature vectors that represent the video, with each feature vector expressing visual information content of one single frame from the sequence of video frames. The second stage then uses the outputs of the first stage and refines or finalizes the surgical phase prediction. In some embodiments, the video of the surgery is analyzed in real-time or near-real-time.

In some embodiments, the trained two-stage SPR module 110 identifies a quality marker in the video at step 1320. In some examples, a quality marker refers to an action or situation that may result in an adverse event. For example, when the surgery is expected to include multiple, sequential surgical phases, then the absence or omission of a surgical phase may indicate a missed step that can create an adverse event later in the surgery. Specifically, in laparoscopic cholecystectomy, the surgical phase of achieving the Critical View of Safety (CVS) is recommended for minimizing the risk of Bile Duct Injury (BDI). If the two-stage SPR module 110 detects that the surgical phase of achieving the CVS was skipped, then the quality marker may be an indication that a specific surgical phase was omitted or skipped and/or that risk of BDI is increased.

Another quality marker may be the time spent in a surgical phase. In the training of the two-stage SPR module 110, the two-stage SPR module 110 learns about the relationship between time spent in each surgical phase and potential outcomes. Some potential outcomes are adverse and knowing the relationship between the time spent and these potential outcomes can be used to predict the potential adverse outcome. The two-stage SPR module 110 learns average time periods that are specific to hospital location, surgeon, type of surgery, etc. If the time spent in a surgical phase is greater than an average time spent in the surgical phase or exceeds a predetermined threshold relating to the time spent, then the two-stage SPR module 110 may consider the amount of time spent in the surgical phase as a quality marker, which might be associated with an increased risk of a potential adverse outcome. Similarly, if the time spent in a surgical phase is much less than average, then the two-stage SPR module 110 may consider the reduced time spent as a quality marker, which might be associated with an increased risk of a potential adverse outcome. Spending a short amount of time or too much time in one surgical phase is an indication that the phase may not have been completed correctly and/or that there are complications in completing the surgical phase.

Quality markers also include markers indicating that the surgery is progressing as expected and as scheduled. Another example of a quality marker is the identification of a specific tool used in surgery. When the use of a tool in surgery is indicative of an abnormal event or when the use of the tool is associated with later adverse effects, then the two-stage SPR module 110 may associate the identification of the tool as a quality marker that is associated with an increased risk of a potential adverse outcome. There are many quality markers that can be identified by the two-stage SPR module 110 and are not limited to the identification of a surgical phase, timing relating to each surgical phase, and use of tools.

In some embodiments, feedback regarding the identified quality marker is output at the step 1325. The feedback is dependent upon the quality marker itself. If the quality marker is associated with a real-time event/circumstance that may later cause a potential adverse event, the two-stage SPR module 110 may provide feedback regarding the identification of the quality marker and/or identification of the potential adverse event. In some embodiments, the model 110 provides an indication (e.g., audio, tactile, visual alert/indication) to a user of the system, wherein the indication indicates the existence of the quality marker and/or the potential adverse event. If the quality marker is associated with the duration of surgical phase(s), the two-stage SPR module 110 may provide feedback re predicted surgery duration. In some embodiments, the model 110 provides an indication (e.g., audio, tactile, visual alert/indication) to a user of the system, wherein the indication indicates the predicted surgery duration, predicted procedure/surgery completion, the need to reschedule a later scheduled surgery for a later time when the surgery duration is extending beyond a scheduled surgery duration, the need to reschedule a later scheduled surgery for an earlier time when the surgery duration is shorter than a scheduled surgery duration, indicate other staffing needs, etc. In some embodiments, the two-stage SPR module automatically reports to an electronic scheduling system, which then reschedules downline surgeries. There are many types of feedback that can be output.

The AI system 100, which recognizes surgical phases, may be used for many important tasks like quality measures, adverse events recording and analysis, education, statistics, surgical performance evaluation and more. Use of the system 100 during surgery enables real-time monitoring and assisted decision making, which may increase safety and improve patient outcomes. For example, a real-time assistive system, such as the system 100, may alert the surgeon to an incorrect plane of dissection, a wrong maneuver, or an upcoming complication. In some embodiments, the system 100 is used as a context-aware decision support system by providing early warnings in case of misorientation or other unexpected events. As a specific example in LC, and as previously noted, achieving the Critical View of Safety (CVS) is the recommended strategy for minimizing the risk of Bile Duct Injury (BDI). The system 100 detecting and verifying that CVS has been achieved is an improvement to the technical field of computer vision AI systems. In some embodiments, the system 100 optimizes operating room (OR) utilization and staff scheduling as well as provides administrative assistance by analyzing the progress of an operation and more accurately predicting the time required for procedure completion.

Non-real-time uses of the AI system 100 includes analyzing LC videos to provide valuable data to evaluate and track trainees' surgical skill level over time, and to identify correlations between specific events occurring during a procedure and outcomes, such as successful conclusion of the procedure. In some embodiments, AI system 100 enables finer-grained analysis of time taken for procedures, providing insights that augment systems that predict surgical duration and hence aid operating room planning. In some embodiments, and as described with respect to the system 100, the two-stage SPR module 110 is incorporated into the surgical system 105 to provide real-time or near-real-time feedback regarding surgeries.

Such real-time or near-real-time use may play a role in active monitoring to improve patient safety, by providing the surgeon with indications of the successful conclusion of the various surgical phases and alerting if there might be potential issues with the surgical view or dissection plane. For instance, if the described AI system 100 was not able to satisfactorily recognize the CVS, an alert could be generated to prompt re-evaluation of their perception of the anatomy, before proceeding to the clipping and cutting phase, which is irreversible. Although the overall complication rate and bile duct injury in LC is very low, a system 100 may improve safety in teaching departments where junior staff are undergoing training. Likewise, similar systems could aid real-time decision making, such as whether to proceed with laparoscopy, to change the surgical technique (i.e., retrograde dissection or subtotal cholecystectomy), to convert to open surgery, to drain only, or to abort the procedure. In some embodiments, AI system 100 may be used in other types of laparoscopic procedures, such as solid organs surgery, and also used in different types of open surgery.

Generally, the image quality of frames in surgical videos has significant variability owing to movement during video capture, which renders AI analysis more challenging. In addition, anatomical structures and surgical planes are often hidden under fatty tissue and must be exposed before yielding a clear field of view for an AI system's interpretation. In order to improve on accuracy, the model 110 is trained with videos representing real-world variability across anatomy, surgeon's technique, operative tools, surgical complexity, and intraoperative complications. In particular, the example detailed herein included often-encountered complex procedures such as those requiring retrograde dissection, conversion to open procedure, and cholecystitis of varying severity.

In some embodiments, the AI system 100 is used for surgical skill assessment, efficient OR schedule planning, and to assist the surgeon in avoiding technical errors, alert them to imminent complications, and provide real time information to be used for better decision making.

The method and system may be altered in a variety of ways. For example, ResNet50 is described as the deep convolutional neural network trained to classify each frame's associated surgical phase, but other deep convolutional neural networks may be used instead of ResNet50, such as VGG-16, Inceptionv3, and EfficientNet, among others.

Generally, the combination of the first stage and second stage modules results in an improvement to the technical field of computer vision assisted surgery and/or the technical field of computer vision AI systems. The first stage extracting visual features from single frames, without any temporal context, paired with the second stage that aggregates temporal information from neighboring frames results in a model that is designed to leverage the temporal context in the video for improved phrase prediction.

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

The disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

It is understood that variations may be made in the foregoing without departing from the scope of the disclosure. Furthermore, the elements and teachings of the various illustrative example embodiments may be combined in whole or in part in some or all of the illustrative example embodiments. In addition, one or more of the elements and teachings of the various illustrative example embodiments may be omitted, at least in part, and/or combined, at least in part, with one or more of the other elements and teachings of the various illustrative embodiments.

Any spatial references such as, for example, “upper,” “lower,” “above,” “below,” “between,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” “front-to-back,” etc., are for the purpose of illustration only and do not limit the specific orientation or location of the structure described above.

In several example embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims. In the claims, means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What we claim is:

1. A system configured to automatically identify a surgical phase of a surgery captured in a video of the surgery, the system comprising a non-transitory computer readable medium having stored thereon a plurality of instructions:
wherein the surgery comprises a plurality of sequential phases;
wherein the video comprises a sequence of video frames; and
wherein the instructions are executed with one or more processors so that the following steps are executed:
receiving, by a two-stage surgical phase recognition (“SPR”) module, the video of the surgery;
wherein the two-stage SPR module comprises a first stage and a second stage;
extracting, using a neural network that forms the first stage, visual information content of a single frame based on the single frame; and
identifying, using a multi-stage temporal convolution network that forms the second stage, surgical phases captured in the frames of the video based on the visual information content from the first stage; and
wherein the two-stage SPR module has been trained using videos of surgeries involving complex anatomies and videos of surgeries involving adverse events.

2. The system of claim 1,
wherein output of the neural network comprises a sequence of feature vectors that represent the video, with each feature vector expressing visual information content of one single frame from the sequence of video frames;
wherein the sequence of feature vectors is an input to the multi-stage temporal convolution network; and
wherein the multi-stage temporal convolution network comprises temporal convolution layers with a dilation rate that increases across layers to capture temporal connections between the sequence of feature vectors.

3. The system of claim 2,
wherein the neural network extracts visual information content of the single frame based on the single frame and is temporal-agnostic; and
wherein the multi-stage temporal convolution network is non-casual.

4. The system of claim 2,
wherein the multi-stage temporal convolution network comprises a plurality of stages; and
wherein each stage in the plurality of stages comprises a plurality of temporal convolution layers with a dilation rate that increases across the layers of that stage.

5. The system of claim 2,
wherein the video of the surgery is created at a first location;
wherein the instructions are executed with the one or more processors so that the following step is also executed:
before receiving the video of the surgery, retraining a plurality of last prediction layers of the neural network on a dataset associated with the first location.

6. The system of claim 1,
wherein the system further comprises a camera; and
wherein the instructions are executed with the one or more processors so that the following step is also executed:
creating, using the camera, the video of the surgery.

7. The system of claim 1, wherein identifying the surgical phases occurs in real-time or near-real-time.

8. A system configured to identify an adverse event during a surgery based on a video of the surgery, the system comprising a non-transitory computer readable medium having stored thereon a plurality of instructions:
wherein the surgery is on a patient and comprises a plurality of sequential surgical phases;
wherein the video comprises a sequence of video frames;
wherein the instructions are executed with one or more processors so that the following steps are executed:
receiving, by a two-stage surgical phase recognition (“SPR”) module, the video of the surgery;
wherein the two-stage SPR module comprises a first stage and a second stage;
extracting, using a neural network that forms the first stage, visual information content of a single frame based on the single frame;
identifying, using a multi-stage temporal convolution network that forms the second stage, surgical phases captured in the frames of the video based on the visual information content from the first stage; and
identifying, using the two-stage SPR module and the identified surgical phases, an adverse event during the surgery;
wherein the adverse event comprises at least one of:
the absence of a surgical phase in the plurality of sequential surgical phases; and
an injury to the patient.

9. The system of claim 8,
wherein output of the neural network comprises a sequence of feature vectors that represent the video, with each feature vector expressing visual information content of one single frame from the sequence of video frames;
wherein the sequence of feature vectors is an input to the multi-stage temporal convolution network; and
wherein the multi-stage temporal convolution network comprises temporal convolution layers with a dilation rate that increases across layers to capture temporal connections between the sequence of feature vectors.

10. The system of claim 8, wherein the instructions are executed with the one or more processors so that the following step is also executed:
in response to the identification of the adverse event, annotating the video to indicate the video includes the adverse event.

11. The system of claim 8, wherein the instructions are executed with the one or more processors so that the following step is also executed:
in response to the identification of the adverse event, automatically providing an indication to a user of the system, wherein the indication indicates the existence of the adverse event.

12. The system of claim 8, wherein identifying the surgical phases occurs in real-time or near-real-time.

13. The system of claim 8,
wherein the neural network extracts visual information content of the single frame based on the single frame and is temporal-agnostic; and
wherein the multi-stage temporal convolution network is non-casual.

14. A method of identifying an adverse event using a video of a surgery and a two-stage surgical phase recognition (“SPR”) module, the method comprising:
receiving, by the two-stage SPR module, a video of the surgery;
wherein the video comprises a sequence of video frames;
wherein the surgery is on a patient and comprises a plurality of sequential surgical phases; and
wherein the two-stage SPR module comprises a first stage and a second stage;
extracting, using a neural network that forms the first stage, visual information content of a single frame based on the single frame;
identifying, using a multi-stage temporal convolution network that forms the second stage, surgical phases captured in the frames of the video based on the visual information content from the first stage; and
identifying, using the two-stage SPR module and the identified surgical phases, an adverse event during the surgery; and
wherein the adverse event comprises at least one of:
the absence of a surgical phase in the plurality of sequential surgical phases; and
an injury to the patient.

15. The method of claim 14,
wherein output of the neural network comprises a sequence of feature vectors that represent the video, with each feature vector expressing visual information content of one single frame from the sequence of video frames;
wherein the sequence of feature vectors is an input to the multi-stage temporal convolution network; and
wherein the multi-stage temporal convolution network comprises temporal convolution layers with a dilation rate that increases across layers to capture temporal connections between the sequence of feature vectors.

16. The method of claim 15,
wherein the video of the surgery is created at a first location; and
wherein the method further comprises before receiving the video of the surgery, retraining a plurality of last prediction layers of the neural network on a dataset associated with the first location.

17. The method of claim 16, further comprising, in response to the identification of the adverse event, annotating the video to indicate the video includes the adverse event.

18. The method of claim 14, further comprising, in response to the identification of the adverse event, automatically providing a notification regarding the existence of the adverse event.

19. The method of claim 18,
wherein identifying, using the two-stage SPR module and the video, the surgical phases occurs in real-time or near-real-time; and
wherein automatically providing a notification regarding the existence of the adverse event occurs in real-time or near-real-time.

20. The method of claim 14, further comprising, before receiving the video of the surgery, training the two-stage SPR module;
wherein training the two-stage SPR module comprises:
collecting a dataset of surgery videos;
annotating the frames of the surgery videos with one surgical phase from the plurality of sequential surgical phases;
annotating at least a portion of the frames of the surgery videos with one adverse event from a plurality of adverse events;
creating a training set comprising a portion of the annotated surgery videos; and
training the neural network using the training set.
